(19) United States
(12) Patent Application Publication
(10) Pub. No.: US 2025/0040959 A1
(43) Pub. Date: Feb. 6, 2025

(54) A NEW CANNULA PROTECTOR

(71) Applicant: resQdevices Pty Ltd, Eveleigh, New South Wales (AU)

(72) Inventors: Roger Price, Hornsby Heights (AU); Suvra Price, Eveleigh (AU)

(21) Appl. No.: 18/710,743

(22) PCT Filed: Nov. 21, 2022
(86) PCT No.: PCT/AU2022/051394

§ 371 Date: May 16, 2024

(30) Foreign Application Priority Data
Nov. 19, 2021 (AU) 2021903746

(51) Int. Cl. A61B 17/34

(52) U.S. Cl. CPC A61B 17/3421

(57) ABSTRACT

A cannula protector is described. The retainer has a peripheral frame for sur-rounding at least a portion of a periphery of a cannulation site, a cover for facilitating cannulation site inspection, and a cover retention strap extending between portions of the substantially peripheral frame. It can include a hinge integral with the arch to facilitate the cover moving between the open and closed positions.

### PRIORITY

This application is a National Stage Application from PCT/AU2022/051394 filed Nov. 21, 2022, which claims priority from AU 2021903746 filed on Nov. 19, 2021, the entire contents of which are incorporated herein by referring to them in this statement. The instant application claims priority hereby to both PCT/AU2022/051394 and Australian Application AU 2021903746.

### TECHNICAL FIELD

The present technology relates generally to cannula protectors.

### BACKGROUND

Cannulae are very useful devices for introducing fluid to veins and arteries, but they are known to fail. Failure seems to arise from the tip of the cannula bumping up against the tunica intima (lining) of the vein. This generally results in infiltration (fluid going into the tissue rather than the vein), phlebitis (inflammation) and other issues.

Being able to see a cannulation site, and therefore any associated redness and swelling that may arise, are good ways to interrupt at least some of the abovementioned complications from mechanical bumps delivered to the cannula. Known cannula protectors do provide visibility and access to a cannulation site, but do not provide suitable protection for difficult or different cannulation sites. Three known difficult cannulation sites include: the inside of the elbow (cubital fossa); the back (dorsum) of the hand; and the thumb side of the wrist (cephalic).

Known cannula protectors also are heavy, expensive and overcomplex.

Others are too simple and inflexible, and require too much operator input, relying on the skill of the physician to secure the retainer.

The present invention seeks to ameliorate one or more of the above mentioned disadvantages or at least provide a new alternative product.

### SUMMARY

Broadly, the present technology provides a cannula protector having a peripheral frame on which a domed cover is pivotally mounted, and a retaining element at one end to retain the domed cover in a closed position.

Broadly, the present technology also provides a cannula protector with a substantially open-ended frame which includes a pair of frame elements extending, in use, generally along either side of a vein, and including an arm to join the end portions of one end of the pair of frame arms.

Broadly, the present technology provides a cannula protector with at least two spaced-apart frame elements disposed in use, either side of a fluid-carrying vessel.

In accordance with one aspect of the present technology there is provided a cannula protector including:
a frame including one or more frame elements for engaging with one or more peripheral areas of a cannulation site and an arch connecting the frame elements at a proximal end of the frame;
a cover for facilitating inspection of the cannulation site, the cover being movable between an open position distal the cannulation site, and a closed position over the cannulation site,
a hinge integral with the arch to facilitate the cover moving between the open and the closed positions, the hinged arch providing a biasing means for the cover which biases the cover to the open position, and
at least one cover retention element extending between two portions of two frame elements to retain the cover,
wherein the arch elevates the hinge at the proximal end to provide frame clearance to provide room for cannula lines and to keep pressure off the vein or artery.

In one embodiment, the arch acts as a torsion bar. In one embodiment the frame includes a hinge to facilitate the cover moving between the open and the closed positions.

In one embodiment the cover is removable to move between the open and the closed positions.

In one embodiment the cover is detachable from the hinge.

In one embodiment a proximal portion of the cover is mounted to the hinge to pivot between the open and the closed positions.

In one embodiment the frame is in the form of a horseshoe-style frame, wherein the two spaced-apart frame arms are connected at a base or proximal end, and the spaced-apart frame arms are open at their distal end.

In one embodiment the cover hinge is disposed at the base or proximal end of the frame so that the cover moves clear of the distal end when it moves to the open position to improve access to the cannulation site.

In one embodiment the base includes an arch to elevate the hinge at the base or proximal end. This is advantageous because it provides frame clearance to provide room for cannula lines and to keep pressure off the vein or artery.

In one embodiment the arch is configured to twist along its length during opening to the open position or during closing to the closed position. This one arrangement further advantageously provides a biasing means for the cover, which biases the cover to the open position.

In one embodiment the cover retention element is resilient to facilitate removal and installation of the cover.

In one embodiment the cover retention element is extensible to facilitate stretching over the end of the cover during the act of closing the cover in the closed position.

In one embodiment the cover retention element is arched to facilitate patency of an associated vein or artery.

In one embodiment the cover retention element is a rubber material.

In one embodiment the hinge is a rubber material.

In one embodiment the hinge is TPE or silicone or suitable material.

In one embodiment the one or more frame elements include at least two opposed frame arms spaced apart to engage in use with a patient's skin on either side of a vein or artery.

In one embodiment the substantially peripheral frame includes at least two opposed frame arms extending generally along a vein.

In one embodiment the substantially peripheral frame includes a flexible element between the two opposed frame arms.

In one embodiment the flexible element is a strap which provides a degree of relative movement between the distal ends of the two frame arms. This is advantageous in a situation where the cannulation site has multiple contours and a high degree of conformity to difficult contouring would be useful.

In one embodiment the flexible element is arched, to cooperate with a domed section of the cover, which facilitates patency of an associated vein or artery, and to keep clear of a cannula line when in use.

In one embodiment the substantially peripheral frame has an open distal end, distal the proximal end.

In one embodiment the strap is detachable from the frame.

In one embodiment the webbing element is connected to the distal ends of the frame by a weakened portion. Some alternative arrangements of the webbing element are detachable by cutting with scissors.

In one embodiment there is also provided a bumper bar at the distal end for inhibiting damage to the cannula, cannulation site, inhibiting inadvertent access to the cover, or to notify a user of inappropriate movements, nearby hard surfaces, or foreign objects.

In one embodiment the bumper bar is detachably mounted on the distal end of the frame.

In one embodiment the bumper bar is snap fitted onto the distal end of the frame.

In one embodiment the frame includes a bumper bar mount.

In one embodiment the bumper bar mounts are sockets or plugs, or cooperating snap clips, press studs, or like elements.

In one embodiment there are provided a plurality of body strap mounts, spaced along the opposed frame arms, so that a body strap can be fastened to the frame arms for fastening to a patient's limb or body part.

In one embodiment the body strap mounts are bosses or posts for receiving strap holes in a strap.

In one embodiment there are provided six body strap mounts, three along each frame arm.

In one embodiment the frame arms are arcuate, for facilitating mounting to different structural features on the body of the user.

In one embodiment body straps are provided to mount the frame onto a user.

In one embodiment the body straps are TPE.

In one embodiment the body straps include a plurality of holes spaced along the body strap to facilitate adjustable mounting onto the bosses.

In one embodiment the bosses or posts include cutaways for retaining the straps, to form a hook.

In one embodiment the frame elements include ventilation grooves.

In one embodiment the frame elements' ventilation grooves are skarf-like features to facilitate bending to conform to various body forms.

In one embodiment the ventilation grooves extend transversely to the direction of the frame arms so that sweat drains across the arms and away from the cannulation site.

In one embodiment the spaced-apart frame arms include flat upper surfaces to provide surface area for a body strap tape to fasten.

In one embodiment the body strap tape is an adhesive tape.

In one embodiment the cover is transparent to facilitate ready visualisation and inspection of the cannulation site.

In one embodiment the cover is domed to accommodate the cannula and to inhibit and deflect impacts.

In one embodiment the cover is connected to the frame at the arched end by a hinge.

In one embodiment the cover is removable from the frame at the hinge end so that the cover in the open position is entirely disconnected from, and distal, the frame.

In one embodiment the cover includes a cover archway at the distal end to facilitate patency of a relevant vein.

In one embodiment the cover archway receives the strap to retain the cover onto the frame at the distal end.

In one embodiment the cover archway includes a strap retainer to retain the distal end strap.

In one embodiment the cover is assembled to the frame by being retained under two arches, one at one end, and one at the other, each one being in the form of the archway above described at the distal end.

In one embodiment the strap retainer is in the form of a retaining saddle which keeps the end strap in a saddle channel between two saddle walls.

Broadly, the present technology provides a cannula protector with a substantially peripheral frame, which includes a plurality of strap anchors spaced along the peripheral frame.

Broadly, the present technology provides a cannula protector with a substantially peripheral frame for substantially surrounding a cannulation site, and a bumper element disposed at a distal end for providing bump notifications to a wearer.

In one embodiment there is provided a lock arrangement provided for the cover.

In one embodiment the lock system includes a lock receiver and a tab.

In one embodiment there the lock receiver is disposed on the cover and is in the form of a guide, and can take the form of a rib, or a pair of ribs, a knurled section, or a groove.

In one embodiment the lock receiver is provided to more securely lock the cover to the peripheral frame elements.

In one embodiment the lock receiver also minimizes lateral drift of the cover relative to the peripheral frame elements.

In one embodiment the lock receiver is a hole in the retainer neck, in the base of the saddle channel, which is configured to receive the tab, which is disposed on the retainer strap.

In one embodiment the lock system further includes one or more further lock receivers disposed on the cover.

The further lock receivers and are configured to receive the retainer strap and are configured to inhibit the cover moving laterally and axially relative to the elongate elements.

In one embodiment the further lock receivers are also configured to inhibit removal of the strap from the saddle in that they securely receive the strap and retain it.

In one embodiment, to facilitate that secure retention the further lock receivers are in the form of notches, disposed in edges of the cover, and the notches include a neck and a bight and arranged such that the strap is inhibited from removal from the bight by the neck.

In one embodiment on the retainer strap is a grab element for use by a user in grabbing the strap. The grab element is in the form of a tab extending from the retainer strap so that the user can more easily lift the retainer strap out of the saddle so that the lock tab is released from the lock receiver and over the saddle wall to release the cover and provide access to the cannulation site.

In one embodiment the cover includes a bumper. The bumper in some embodiments is disposed only at the distal end of the cover.

In the embodiment shown at FIGS. 20 to 24, the bumper extends all around the cover to inhibit cuts and lacerations to hands when handling.

In one embodiment a fastener is provided to fasten the elongate elements to a patient's skin.

The fastener may be in the form of liquid adhesive or glue.

The fastener may also be in the form shown, which is adhesive tape. The adhesive tape is in the form of straps, attached to and extending from the elongate elements, at acute angles. The acute angles are to provide additional security of adhesion to a patient, being a balance between axial security and radial security. The adhesive straps may be provided with one end affixed to the elongate elements at 45 degrees to the axis of those elongate elements.

In one embodiment there is also provided a jig which is configured to provide a repeatable increased height for the dome during installation, wherein the jig includes a spacer element.

In one embodiment the spacer element includes a finger.

In one embodiment the finger includes a foam cylinder.

In accordance with another aspect of the present invention there is provided a method of application of a cannula protector over a cannulation site, the cannula protector including one or more peripheral frame elements with one or more adhesive tapes and backing attached thereto and extending therefrom, an arch connecting the frame elements at a proximal end of the frame elements, a cover hingedly mounted on the to facilitate the cover moving between the open and the closed positions, the hinged arch providing a biasing means for the cover to the open position, and wherein the arch elevates the hinge at the proximal end to provide frame clearance to provide room for cannula lines and to keep pressure off the vein or artery, the method including the steps of:
locating the cover over the cannulation site;
removing the backing from the adhesive tapes;
fastening the adhesive tapes to the skin adjacent the cannulation site.

In one embodiment the method includes the step of extending the adhesive tapes at an angle of 45 degrees to the peripheral elongate frame elements.

In one embodiment the method further includes the step of criss crossing the adhesive tapes. This arrangement helps to provide both axial and radial support against movement.

In one embodiment the method includes inserting a spacer between the dome cover and the peripheral frame elements to provide more space under the domed cover.

A method of application of a cannula protector over a cannulation site, the cannula protector including one or more frame elements, an arch connecting the frame elements at a proximal end of the frame elements, a cover hingedly mounted on the arch, the cover being movable between an open position and a closed position, and a hinge integral with the arch to facilitate the cover moving between the open and the closed positions, the hinged arch providing a biasing means for the cover which biases the cover to the open position, and wherein the arch elevates the hinge at the proximal end to provide frame clearance to provide room for cannula lines and to keep pressure off the vein or artery the method including the steps of:
locating the cover over the cannulation site;
fastening the frame elements to the skin of the patient;
accessing the cannulation site by releasing a cover retention element from the cover and allowing the cover to pivot away from the site.

### Advantages

Advantageously, preferred embodiments provide a cannula protector that is configured to be disposed over a cannula and protect that cannula from one or more of a variety of different kinds of mechanical impacts (pulling, brushing, pressure, bumping etc) that likely cause their failure.

In some embodiments there is an open-ended tunnel aspect to the general shape provided by two arches: a first arched hinge at a proximal end and an archway at the distal end provided by the end of the dome. The general open-ended tunnel shape allows for fluid lines to project (usually from the finger end) and to avoid venous congestion (tourniquet effect) on the heart or proximal end.

Further advantageously, preferred embodiments do provide a clear dome for good visibility to the cannulation site. In addition, the arched frame elements and their material being resilient and strong flexy material, provide a balance between rigidity and flexibility to position the protector in awkward anatomical areas and yet still provide protection to the site.

Other advantages of embodiments include ready access for clinicians and nurses to front of the cannula protector by virtue of it being arched at the distal end. Access to the cannulation site for the patient is inhibited, but also there is ready access by nursing staff to the cannulation site by a simple manipulation of the strap to release the cover. Distal end access being arched allows the device to be placed over fluid lines which project outwardly from the arched end, and then extend along the arm and towards the fingers of a patient's hand.

Even further advantages of preferred embodiments include that there is a balance of strength in the hyperbolic paraboloid surface for protection, and the flexibility inherent in the arched connections made between the spaced-apart frame elements. This facilitates bending and flexibility in 2 planes to protect geometrically difficult sites.

The arch at the proximal end acts as a torsion bar so when the protector comes out of the packet it can be placed open over the IV site and adjusted, then the dome is closed. Also, the dome sits out of the way when the nurse is doing other things-she doesn't have to allocate a finger to holding it open.

Finally, embodiments also advantageously are lightweight, low infection risk, easy to use, cheap to manufacture, and low in complexity.

### Clarifications

In this specification, where a document, act or item of knowledge is referred to or discussed, this reference or discussion is not an admission that the document, act or item of knowledge or any combination thereof was at the priority date:
(a) part of common general knowledge; or
(b) known to be relevant to an attempt to solve any problem with which this specification is concerned.

It is to be noted that, throughout the description and claims of this specification, the word ‘comprise’ and variations of the word, such as ‘comprising’ and ‘comprises’, is not intended to exclude other variants or additional components, integers or steps.

### BRIEF DESCRIPTION OF THE DRAWINGS

In order to enable a clearer understanding, a preferred embodiment of the technology will now be further explained and illustrated by reference to the accompanying drawings, in which:

FIG. 1 is a perspective view of a cannula protector in accordance with an embodiment of the present invention, from a proximal end and above and a right hand side;

FIG. 2 is a perspective view of the embodiment shown in FIG. 1, from a right hand side, and the distal end;

FIG. 3 is a side view of the embodiment shown in FIGS. 1 and 2, with a cover shown in an open position;

FIG. 4 is a side view of the embodiment shown in the FIGS. 1 to 3, the frame elements shown fastened with adhesive tape to an arm of a patient at a cannulation site, and the cover shown in a closed position to protect the cannula (not shown) at the cannulation site;

FIG. 5 is a perspective view from above and a left hand side of a prototype cannula protector made in accordance with FIGS. 1 to 4;

FIG. 6 is an isometric view of another embodiment of the present technology from front in a closed position;

FIG. 7 is a right side elevation of FIG. 6 in a closed position;

FIG. 8 is an isometric view of the embodiment shown in FIG. 6 from front in an open position;

FIG. 9 is a front elevation view of the embodiment shown in FIG. 6 with straps removed;

FIG. 10 is a plan view of the embodiment shown in FIG. 6;

FIG. 11 is a rear elevation view of the embodiment shown in FIG. 6 with straps removed;

FIG. 12 is a left side elevation view of the embodiment shown in FIG. 6;

FIG. 13 is a detail view of the front of the embodiment shown in FIG. 6 with straps removed;

FIG. 14 is a detail view of the embodiment shown in FIG. 10 with straps removed;

FIG. 15 a detail isometric view of front corner, straps removed;

FIG. 16 is a side elevation view of the cannula protector of FIGS. 6 to 15;

FIG. 17 is an isometric view of a dome of the embodiment of FIGS. 6 to 16;

FIG. 18 is an isometric view of a substantially peripheral frame of the embodiment shown in FIGS. 6 to 17;

FIG. 19 is an isometric view of a removed snap fit bumper bar for use in the embodiment shown in FIGS. 6 to 18;

FIG. 20 is an isometric view of a third embodiment of cannula retainer shown in an open position;

FIG. 21 is a plan view of the cannula retainer of FIG. 20 shown in an open position;

FIG. 22 is a typical side elevation view of the cannula retainer of FIG. 20 shown in an open position;

FIG. 23 is an isometric view of the third embodiment of cannula retainer shown in FIG. 20 shown in a closed position without fastening tape;

FIG. 24 is a perspective view of a third embodiment of cannula retainer shown with adhesive tape affixed to the elongate frame elements, with backing tape being removed before mounting on a patient;

FIG. 25 is a side view of a third embodiment of cannula retainer after the backing tape is removed from the adhesive tape and the tapes are affixed to the patient in the X pattern shown; and

FIG. 26 is a side view of the third embodiment of cannula retainer sited in the antecubital fossa; and

FIG. 27 is a perspective view of the third embodiment shown with a temporary jig in place before removal to provide extra height under the dome.

### DETAILED DESCRIPTION OF EXAMPLE EMBODIMENTS

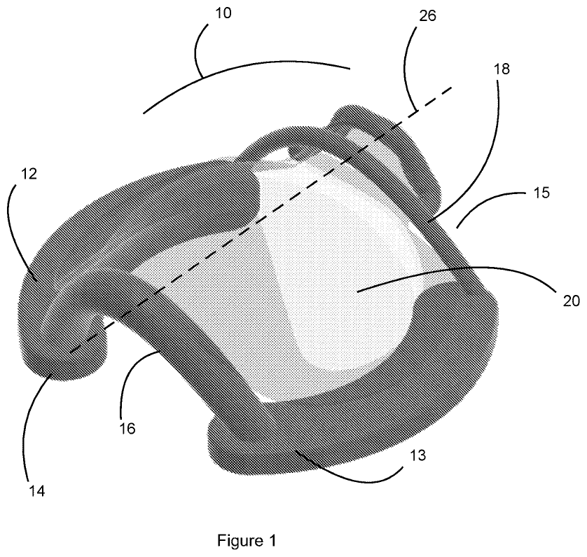

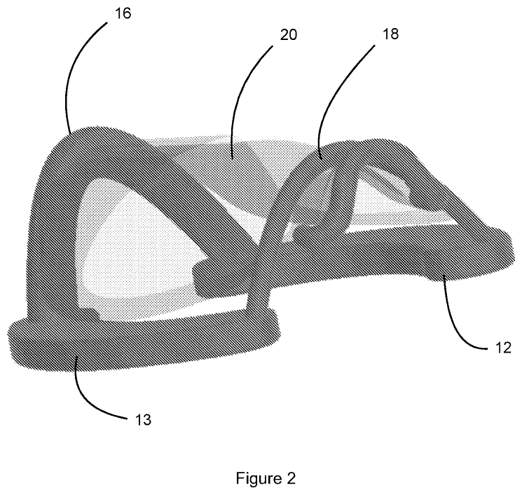

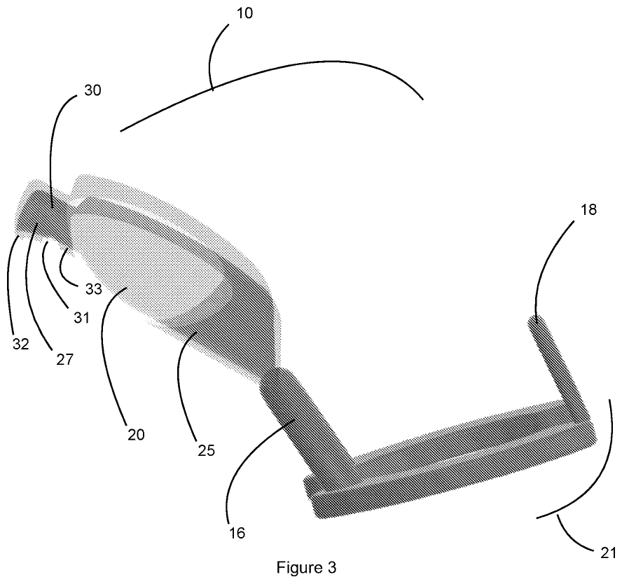

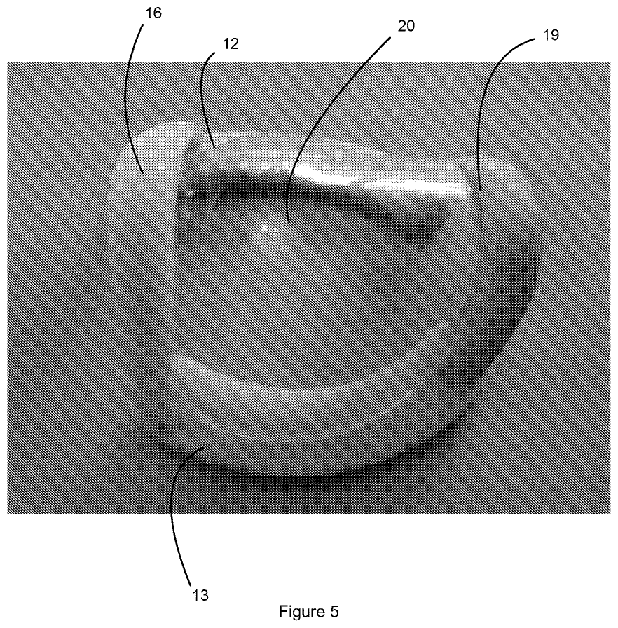

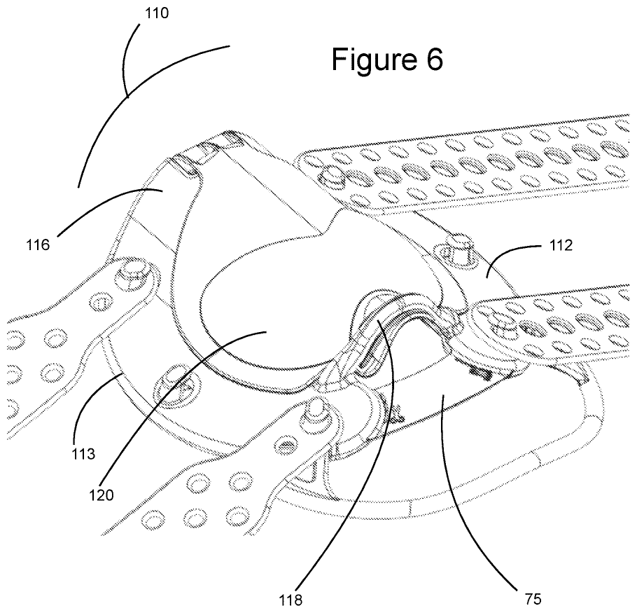

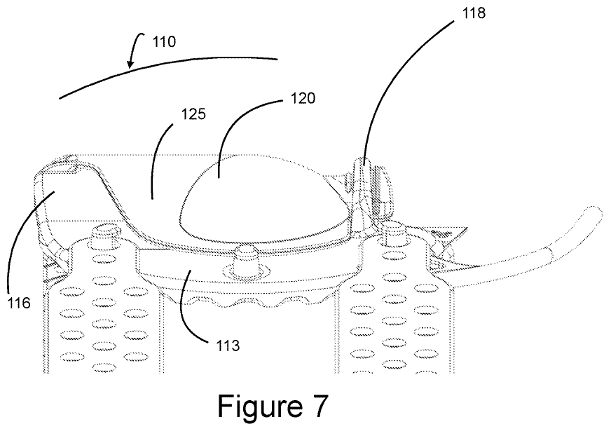

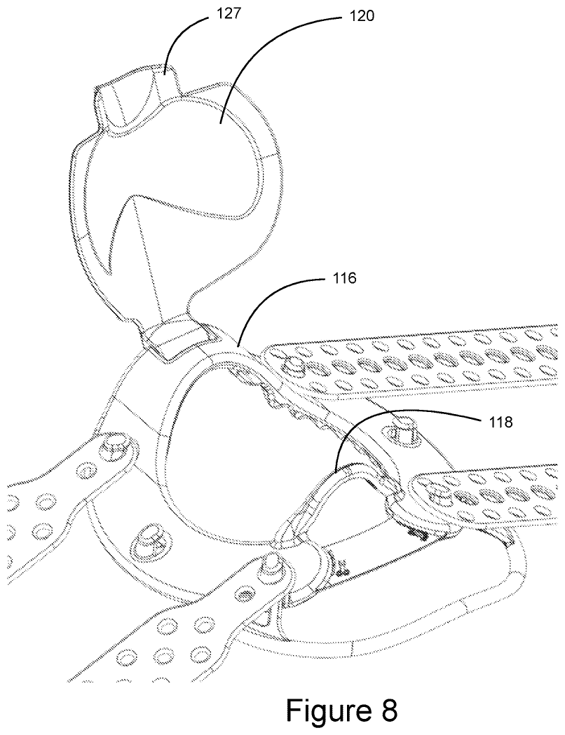

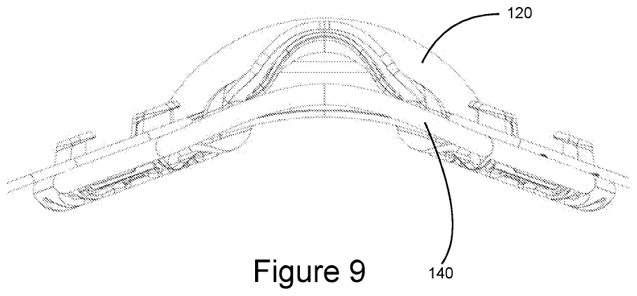

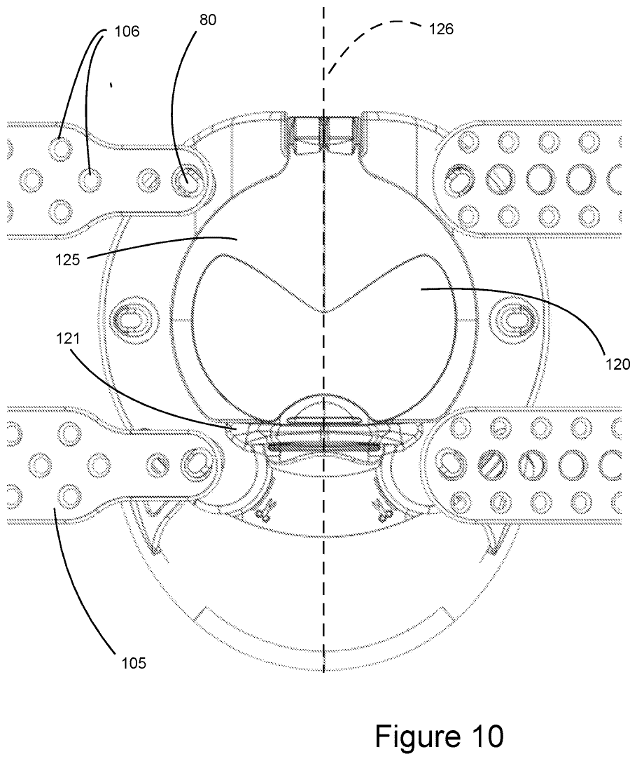

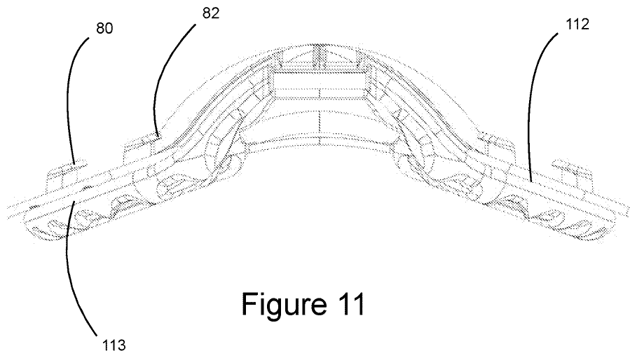

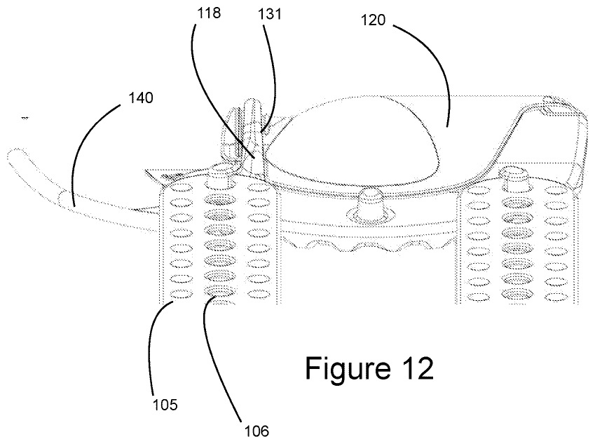

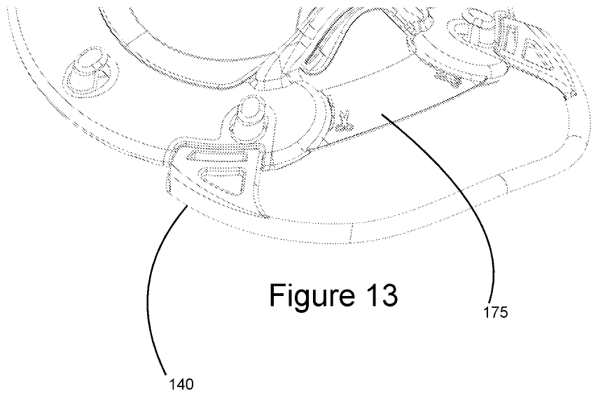

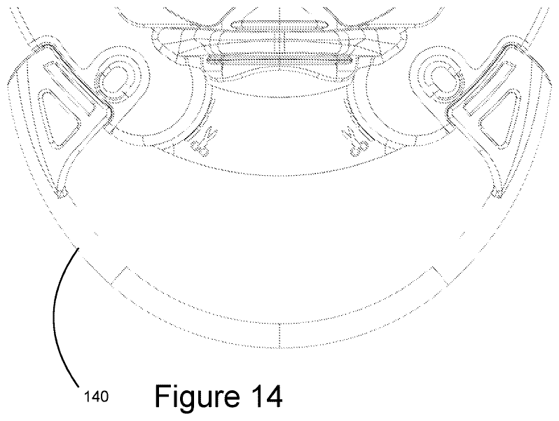

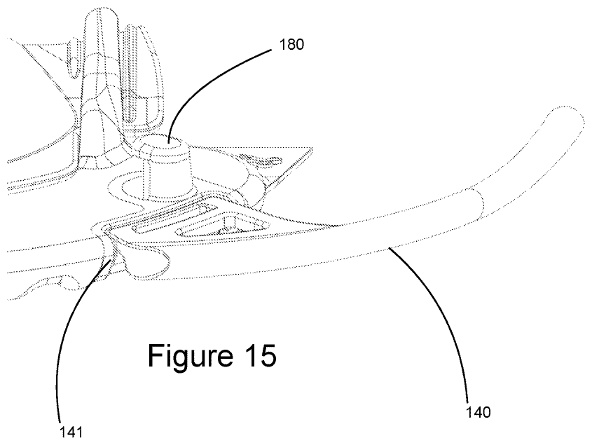

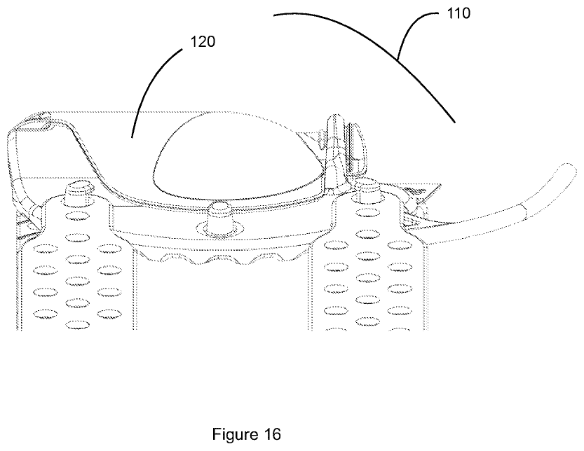

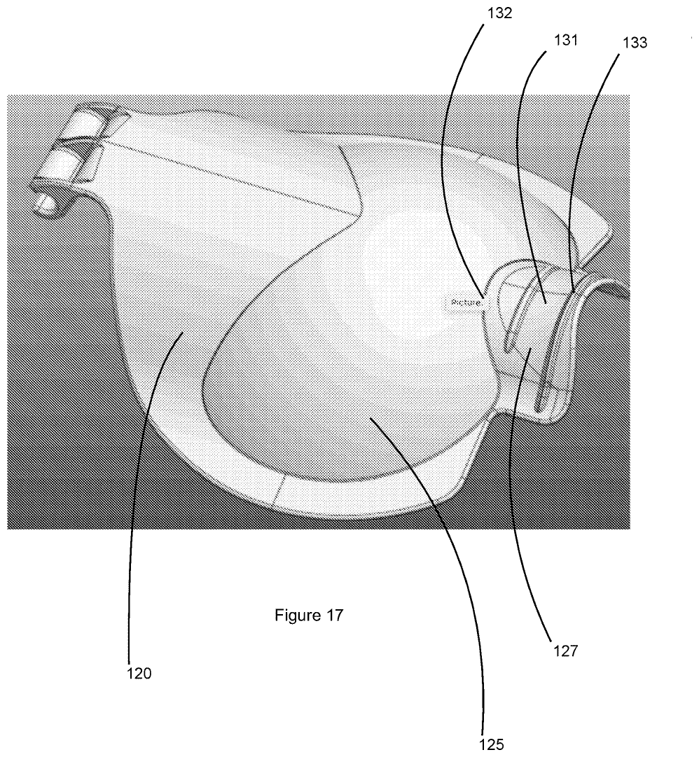

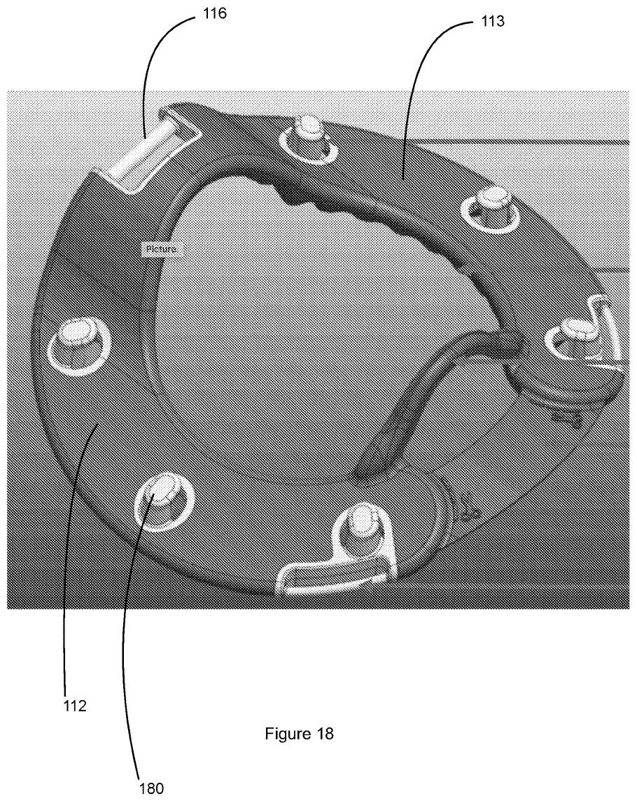

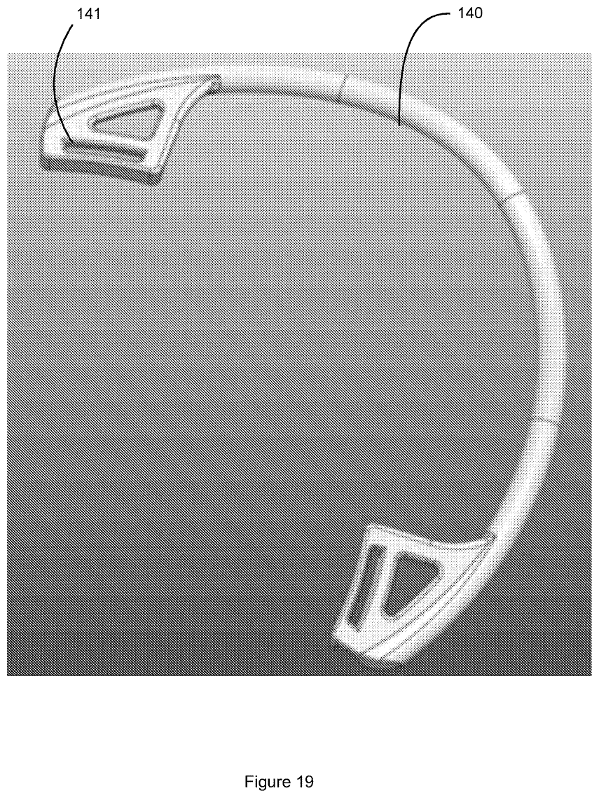

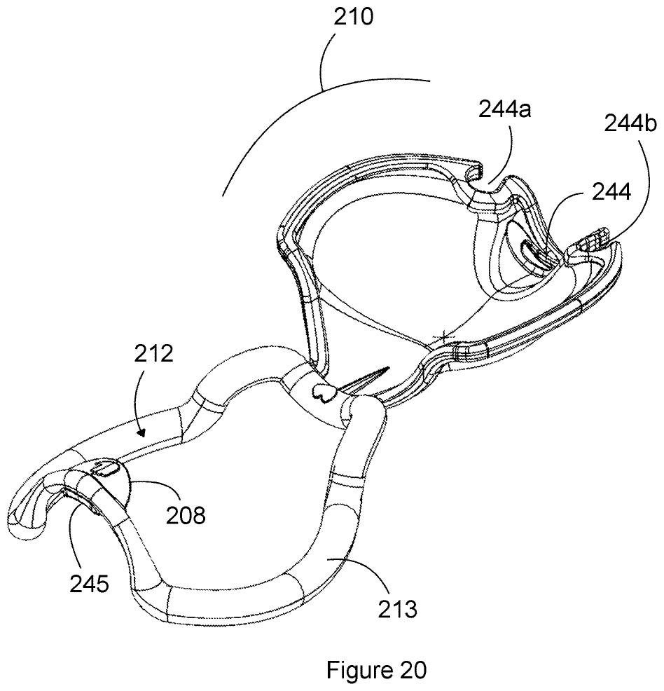

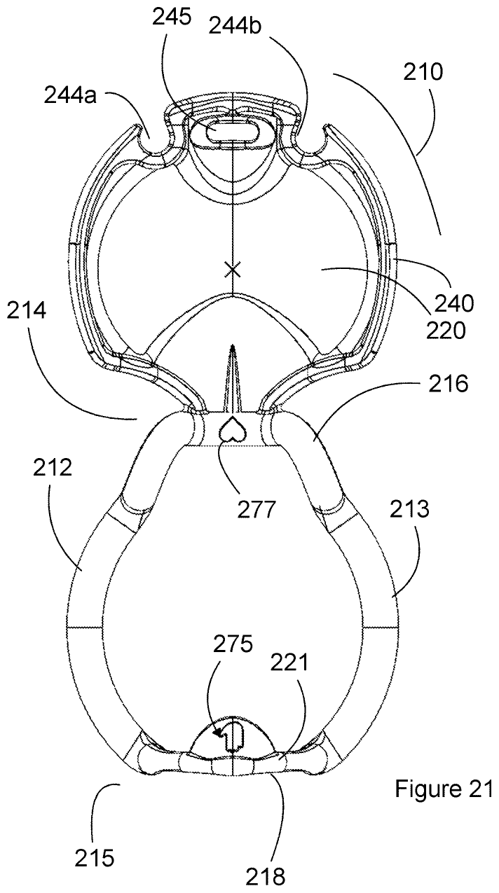

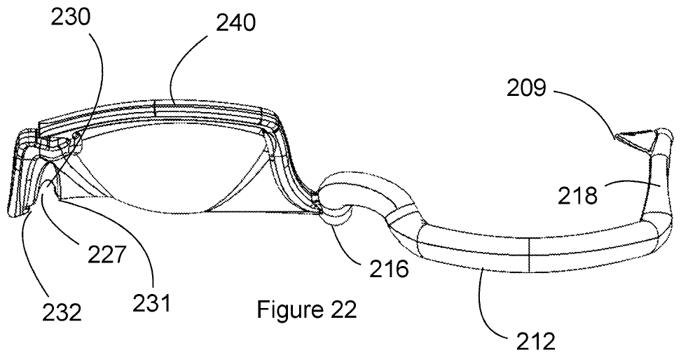

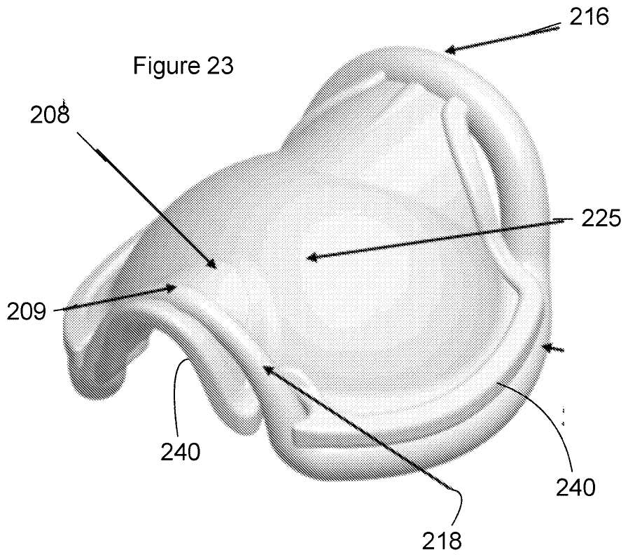

Referring to the drawings there are shown three embodiments of cannula protector, generally indicated at 10 (one embodiment), at 110 (another embodiment) and at 210 (a third embodiment). There are two embodiments described; there are commonalities between the two embodiments, mainly relating to the body strapping and the hinge for the cover. When describing the two embodiments in this specification, and showing them in the drawings, like numerals will denote like parts. For example, a cannula protector of the first embodiment in FIGS. 1 to 5 will be generally indicated at 10, while the cannula protector of the second embodiment shown in FIGS. 6 to 19 will be generally indicated at 110. The cannula protector of the third embodiment shown in FIGS. 20 to 24 will be generally indicated at 210.

The cannula protector 10, 110, 210 includes a frame 11, 111, 211 which includes a pair of opposed peripheral frame elements 12, 13, which are substantially open at their distal ends 15, 17. The peripheral frame elements 12, 13 are spaced apart, and generally elongate and extend along two opposed sides of a periphery, to provide room in between them for a cannula on a patient. The spaced apart peripheral frame elements 12, 13, also are bent into a broad arc, for providing additional space between, for a cannula (not shown). The peripheral frame elements 12, 13 are connected at a proximal end 14, 114, by an archway 16, 116, 216 for generally or substantially rigid spacing and elevating support for a cover 20, 120, 220. At the other end, the distal end 15, 115, the peripheral frame elements 12, 13 are connected by a retainer strap 18, 118, to retain the cover 20, 120, 220 in a closed position. The retainer strap 18, 118 (which, to be clear, could be strap, arm, elbow or other structure) functions as a cover retainer element 21, 121, 221 to retain the cover 20, 120 in the closed position, in which the cover 20 is generally proximal the frame elements 12, 13, to inhibit access to the cannulation site. The cover can adopt an open position in which at least part of it is disposed remote from the peripheral frame elements and the cannulation site.

Also, the retainer strap can be resilient. The retainer strap 18, 118, 218, can also be arched to extend over the top of an arched end of the cover 20.

The peripheral frame elements 12, 13, are elongate in form, and spaced apart, to provide protection for the cannulation site 5 which, when the cannula protector is in use, is disposed underneath the cover 20. The peripheral frame elements 12, 13 are, in one embodiment, round in section, for ease of handling and to reduce hard edges, but may also be flattened on their upper and lower surfaces, to provide (for the upper flat surface) a broad area for to attach adhesive tape 5 to fasten the cannula protector 10 to a patient's limb, hand or head or other area. The lower surface is flattened to reduce point loads on the surface of the patient's skin. The elongate elements 12, 13 are also formed into a broad arc when viewed from above (FIG. 1) to provide an efficient generally circular cannula protection zone, as described above. The elongate elements 12, 13 are also arcuate when viewed from the side to facilitate conformance with concave cannulation sites. The elongate elements 12, 13 are also somewhat flexible so that when the cannulation site is flat, the adhesive tape 50 can bend them to conform to the flat site at least a little.

The peripheral frame elements are formed from TPE or silicone to provide softness and some resilience, axially and radially. This reduces point loads on the skin even when the section is round, since the section can flatten out a little. The peripheral frame elements 12, 13, retainer strap 18, arch 16 and bumper 40 are formed from soft TPE (non-latex) and are all integral with one another. The peripheral frame elements 12, 13 and the other elements may be formed from multiple materials, but in the embodiment shown at FIGS. 20 to 24 they are all the same material and integral with one another.

The cover 20 is a dome 25 or at least curved in one axis 26, to provide additional inherent strength, and to facilitate deflection of inbound objects and like things that might strike and bump the device 10. If inbound objects deflect, then they do not move the device 10 as much, and the cannula is more protected than otherwise.

The dome 25 is a shell with a retaining neck 27 disposed at the distal end 15. The retaining neck 25 is in the form of a saddle 30 with a saddle channel 31 and saddle channel walls 32, 33. The retaining neck 27 includes a retaining flange 42, 142, 242, which is configured to cooperate with the retainer strap 18 to keep the cover 20 in the closed position, in which the since the biasing action of the arch hinge 16, discussed below, facilitates the cover being kept in the open position.

There is also a lock system 243 provided for the cover. The lock system 43, 143, 243 includes a lock receiver 244 and a tab 245. The lock receiver 44 is disposed on the cover 20, and is in the form of a guide 46, 146, 246 and can take the form of a rib, or a pair of ribs, a knurled section, or a groove. The lock receiver 244 is provided to more securely lock the cover 220 to the peripheral frame elements 212, 213.

The lock receiver 44 also minimizes lateral drift of the cover 20 relative to the peripheral frame elements 12, 13. In the embodiment shown, the lock receiver 44 is a hole 45 in the retainer neck 27, in the base of the saddle channel 31, which is configured to receive the tab 45, which is disposed on the retainer strap 18.

The lock system 43, 143, 243 further includes one or more further lock receivers 44a and 44b disposed on the cover 220. The further lock receivers 44a and 44b are configured to receive the retainer strap 218 and are configured to inhibit the cover moving laterally and axially relative to the elongate elements 212, 213. The further lock receivers 44a and 44b are also configured to inhibit removal of the strap from the saddle 30 in that they securely receive the strap 218 and retain it. To facilitate that secure retention the further lock receivers 44a and 44b are in the form of notches 45a and 45b, disposed in edges of the cover 220, and the notches include a neck 46a and a bight 47a and arranged such that the strap is inhibited from removal from the bight 47a by the neck 46a.

On the retainer strap 218 is a grab element 208 for use by a user in grabbing the strap 218. The grab element 208 is in the form of a tab 209 extending from the retainer strap 218 so that the user can more easily lift the retainer strap 218 out of the saddle 230 so that the lock tab 245 is released from the lock receiver 244 and over the saddle wall 232 to release the cover 20 and provide access to the cannulation site.

Also in the embodiment shown, the cover 20, 120, 220 includes a bumper 40, 140, 240. The bumper 40, 140, 240, in some embodiments is disposed only at the distal end of the cover. This arrangement is shown in FIGS. 1 to 5. In the embodiment shown at FIGS. 20 to 24, the bumper 240 extends all around the cover 220 to inhibit cuts and lacerations to hands when handling.

The domed shell is transparent for ease of viewing of the cannulation site. It is formed from a different material from the frame elements etc, since it provides a different function.

In embodiments the biasing of the arch hinge 16 is to the open position to facilitate automatic access to the cannulation site upon release of the cover 20 from the retainer strap 18.

The retainer strap 18 and/or hinge 16 is TPE or silicone or suitable material.

The elongate elements 12, 13, retainer strap 18, and arch 16 are moulded from TPE or silicone material. The durometer of the material is 55 to 60, or 45 to 70 Shore A hardness.

The arch 16, 116 is configured to provide a mount for a cover 20, 120, and the resilient retainer strap 18, 118, 218 provides retention for the cover 20, 120, 220.

Turning back to FIGS. 1 to 5, the arch 16 is a resilient element like, say a TPE or rubber or silicone or foam compound, which includes a groove 19 to receive and mount a proximal edge of the cover 20. In FIGS. 6 to 19, the arch 116 has no such groove 19 to mount the cover 120, but instead, the arch 116 includes a hinge 119 to receive and connect a cooperating portion of the cover 120, so that it can pivot thereabout. The cover 20, 120 could detach from either of the mounts 19, 119, to remove it altogether, so that the open position then involves the removal of the cover 20, 120 and placing it in a position distal the frame 21, 121.

The retainer strap 18 is in one embodiment, inextensible, and in other embodiments, is resilient. The resilient option is provided so that to release the cover 20 from the closed position, a clinician pulls or rolls the strap 18 over a saddle 30 and over the bumper 40, and during the pull, the retainer strap 18, 118, 218 extends a little to stretch over a retaining flange 42 in the form of a saddle wall 32, so that the cover 20 can release from the retainer strap 18. To access the cannulation site, the cover 20 is then pivoted upwardly, against the torsional bias of the archway hinge 16. In some embodiments, the torsional bias of the archway hinge 16 is to the open position so the cover springs open automatically after release of the retainer strap 18.

Body strap mounts 80 are shown disposed on the frame elements 112 and 113, and there are six of them in total, spaced along the frame elements 112, 113. Each one has a keeper 82 which keeps the body strap 105 on the body strap mount 80. There are three rows of cooperating holes 106 in the body straps 105 to maintain a high potential for adjustability.

Bumper 140 is connected to the frame elements 112, 113 by snap fitting clips 141.

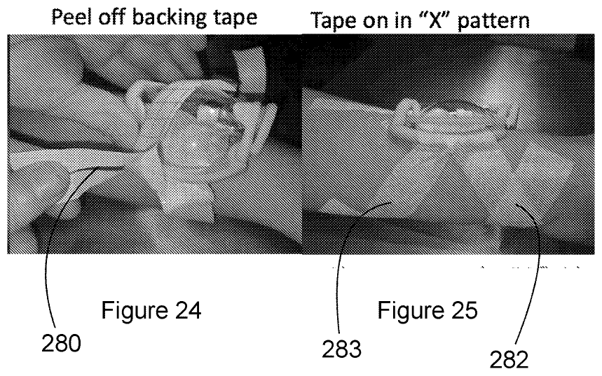

FIGS. 24 and 25 show the backing tape coming off the adhesive tape, and then the adhesive tape being applied to the patient at 45 degrees to the longitudinal axis of the cannula protector. This angle helps to keep the cannula retainer from sliding along the arm longitudinally as well as coming off the arm radially. The tapes cross over each other, which helps to support each one of the tapes with other tapes.

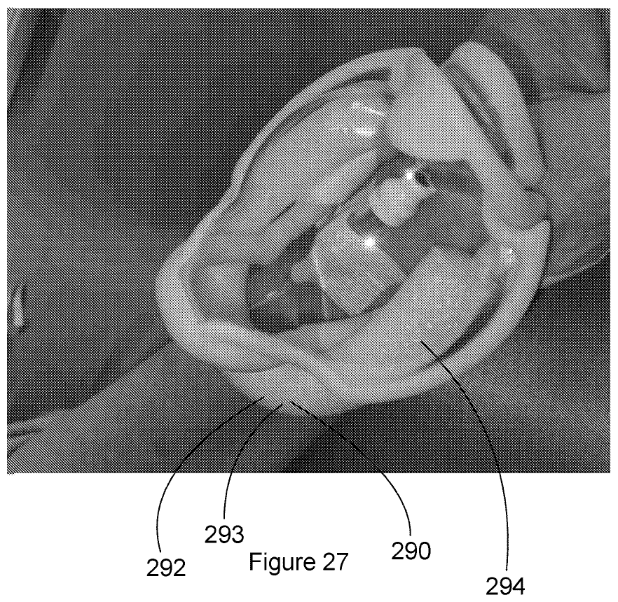

There is also provided a jig 290 which is configured to provide a repeatable increased height for the dome during installation. The jig 290 includes a spacer element 292. The spacer element 292 includes a finger 293. The finger 293 includes a foam cylinder 294, which is configured to be flexible so that one cylinder can extend along one frame element 212, and return along the other frame element 213, to install the cannula protector 210 at the increased height. This can be seen in FIG. 27. The foam cylinder 294 is placed along the elongate elements 212 and 213. To do that, the elongate elements 212, 213 are squeezed together and the foam cylinder 294 holds them in that position, under the dome (which is fairly rigid). This adds height to the dome, and effectively increases the area under the dome. This is useful for mounting on the blade of the forearm. Once the adhesive tapes are applied, the height of the dome is set and the foam cylinder can be removed.

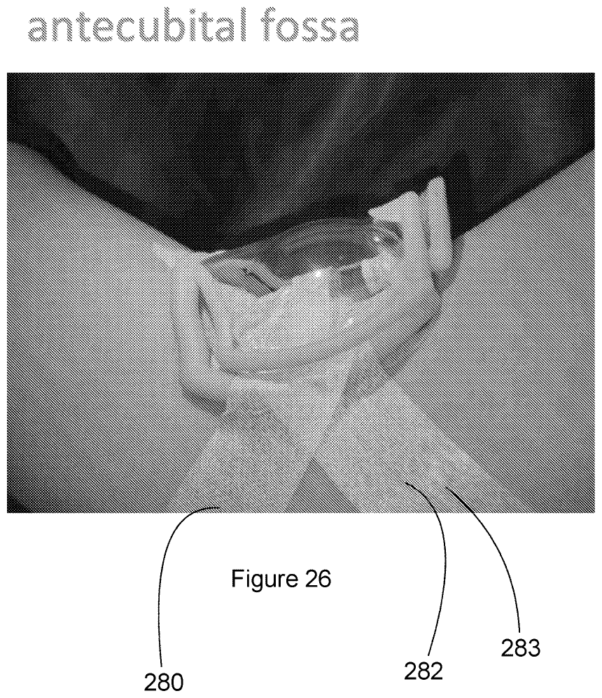

In FIG. 26 the arcuate elements 212, 213 can be seen helping to keep a cannula in a crook of an elbow protected. The arcuate elements 212, 213 help to conform to the crook of the elbow.

A fastener 280 is provided to fasten the elongate elements 213, 212 to a patient's skin. The fastener 280 may be in the form of liquid adhesive or glue. The fastener 280 may also be in the form shown, which is adhesive tape 282. The adhesive tape 282 is in the form of straps 283, attached to and extending from the elongate elements 212, 213, at acute angles. The acute angles are to provide additional security of adhesion to a patient, being a balance between axial security and radial security. The adhesive straps 283 may be provided with one end affixed to the elongate elements 212, 213 at 45 degrees to the axis of those elongate elements 212, 213.

As shown in FIGS. 6 to 19, there are provided strap retaining bosses on the peripheral arms, extending therefrom, to catch and retain straps.

There is provided an orientation indicator system in which there is provided a hand icon 75 at the distal end and a heart icon 77 at the proximal end.

### In Operation

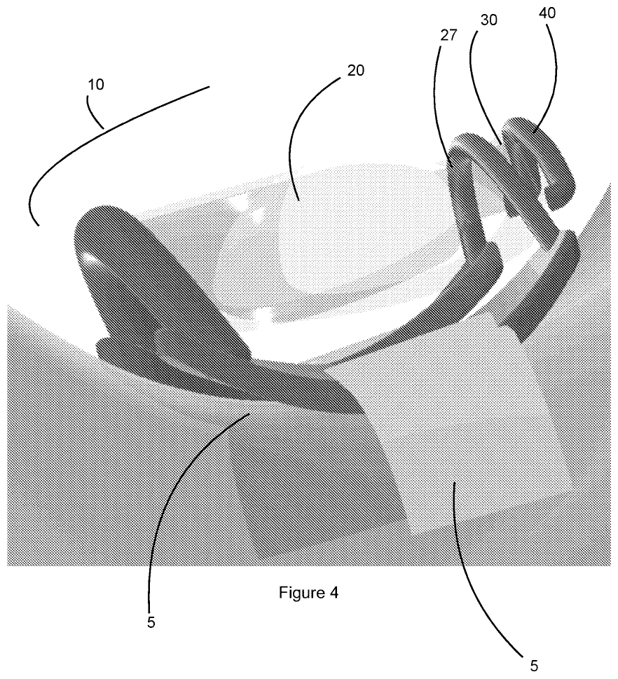

FIG. 4 shows the cannula protector 10 mounted in a protective position in the crook of a patient's elbow. It can be seen that the convex arc of the elongate frame arms 12 and 13 conform to the concave crook. Adhesive tape elements 5 maintain close conformity.

First, the cannula (not shown) is inserted and then the line may be connected. The protector 10 is then disposed on the crook and fastened in position with the adhesive tape 5. The retainer 18 fits over the bumper 40 and sits over the neck 27. The saddle shape of the neck 27 keeps the retainer 18 on the cover, and keeps the cover 20 in the closed position.

In some embodiments the saddle shape of the neck 27 is not pronounced, and could be merely a bottle neck (such that the overall shell shape of the cover would be like that of a wine bottle). In that case, the bumper 40 cooperates with the retainer 18 to keep the cover 20 in the closed position.

In a similar way, the embodiment 110 shown in FIGS. 6 to 19 is applied to the cannulation site. In this embodiment the cannula line, is usually applied after the protector 110 is secured, but if that is not desirable, since it may be already in place, the webbing link 75 is cut, and the bumper bar 140 is removed and then re-attached.

### Clarifications

Modifications and improvements to the invention will be readily apparent to those skilled in the art. Such modifications and improvements are intended to be within the scope of this invention.

1. A cannula protector including:
a frame including one or more frame elements for engaging with one or more peripheral areas of a cannulation site and an arch connecting the frame elements at a proximal end of the frame;
a cover for facilitating cannulation site inspection, the cover being movable between an open position distal the cannulation site, and a closed position over the cannulation site;
a hinge integral with the arch to facilitate the cover moving between the open and the closed positions, the hinged arch providing a biasing means for the cover which biases the cover to the open position, and
at least one cover retention element extending between two portions of two frame elements to retain the cover,
wherein the arch elevates the hinge at the proximal end to provide frame clearance to provide room for cannula lines and to keep pressure off the vein or artery.

2. The cannula protector in accordance with claim 1 wherein the arch acts a torsion bar.

3. (canceled)

4. (canceled)

5. (canceled)

6. (canceled)

7. (canceled)

8. (canceled)

9. (canceled)

10. The cannula protector in accordance with claim 1 wherein the cover retention element is resilient to facilitate removal and installation of the cover.

11. (canceled)

12. The cannula protector in accordance with claim 1 wherein the cover retention element is arched to facilitate patency of an associated vein or artery.

13. The cannula protector in accordance with claim 1 wherein the hinge and cover retention element are TPE.

14. (canceled)

15. The cannula protector in accordance with claim 1 wherein the one or more frame elements include at least two opposed frame arms spaced apart to engage in use with a patient's skin on either side of a vein or artery.

16. (canceled)

17. The cannula protector in accordance with claim 1 wherein the frame arms are arcuate when viewed from above, to provide clearance around the cannulation site.

18. The cannula protector in accordance with claim 1 wherein straps are provided to mount the frame onto a user.

19. (canceled)

20. The cannula protector in accordance with claim 1 wherein there is provided a lock arrangement provided for the cover to more securely lock the cover to the frame elements.

21. The cannula protector in accordance with claim 1 wherein the lock arrangement includes a lock receiver and a tab.

22. The cannula protector in accordance with claim 1 wherein the lock receiver is disposed on the cover and is in the form of a guide, and can take the form of a rib, or a pair of ribs, a knurled section, or a groove.

23. (canceled)

24. The cannula protector in accordance with claim 1 wherein the lock receiver also minimizes lateral drift of the cover relative to the frame elements.

25. The cannula protector in accordance with claim 1 wherein the lock receiver is a hole in a retainer neck, in a base of a saddle channel, which is configured to receive the tab, which is disposed on the cover retention element.

26. The cannula protector in accordance with claim 1 wherein the lock arrangement further includes one or more further lock receivers disposed on the cover, wherein the further lock receivers are configured to receive the cover retention element and are configured to inhibit the cover moving laterally and axially relative to the elongate elements.

27. (canceled)

28. The cannula protector in accordance with claim 26 wherein the further lock receivers are also configured to inhibit removal of the cover retention element from the saddle in that they securely receive the cover retention element and retain it.

29. The cannula protector in accordance with claim 28 wherein to facilitate that secure retention the further lock receivers are in the form of notches, disposed in edges of the cover, and the notches include a neck and a bight and arranged such that the cover retention element is inhibited from removal from the bight by the neck.

30. The cannula protector in accordance with claim 29 wherein on the cover retention element is a grab element for use by a user in grabbing the cover retention element.

31. The cannula protector in accordance with claim 30 wherein the grab element is in the form of a tab extending from the cover retention element so that the user can more easily lift the cover retention element out of the saddle so that the lock tab is released from the lock receiver and over the saddle wall to release the cover and provide access to the cannulation site.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. A method of application of a cannula protector over a cannulation site, the cannula protector including one or more frame elements, an arch connecting the frame elements at a proximal end of the frame elements, a cover hingedly mounted on the arch, the cover being movable between an open position and a closed position, and a hinge integral with the arch to facilitate the cover moving between the open and the closed positions, the hinged arch providing a biasing means for the cover which biases the cover to the open position, and wherein the arch elevates the hinge at the proximal end to provide frame clearance to provide room for cannula lines and to keep pressure off the vein or artery, the method including the steps of:
locating the cover over the cannulation site;
fastening the frame elements to the skin of the patient;
accessing the cannulation site by releasing a cover retention element from the cover and allowing the cover to pivot away from the site.
